(19) United States
(12) Patent Application Publication
(10) Pub. No.: US 2017/0328919 A1
(43) Pub. Date: Nov. 16, 2017

(54) BLOCKADE OF TIM-1 PATHWAYS and P-SELECTIN PATHWAYS IN TREATMENT OF NEUROINFLAMMATORY DEGENERATIVE DISEASE

(71) Applicant: Leuvas Therapeutics, Mountain View, CA (US)

(72) Inventor: Gabriela Constantin, San Floriano (IT)

(21) Appl. No.: 15/592,833

(22) Filed: May 11, 2017

Related U.S. Application Data
(60) Provisional application No. 62/334,986, filed on May 11, 2016.

(51) Int. Cl. G01N 33/68; C12Q 1/68; A61K 38/00; A61K 45/00; C12N 15/85

(52) U.S. Cl. CPC G01N 33/6896; A61K 45/00; C12N 15/8509; C12Q 1/6883; A61K 38/005; C12Q 2600/112; C12Q 2600/156; C12N 2015/8545

(57) ABSTRACT

Methods for treating, preventing and reducing the progression of neurodegenerative and neurological diseases, including, without limitation Alzheimer's disease, are provided. The methods of the invention inhibit one or both of TIM-1 and P-selectin function and reduce leukocyte and platelet cell adhesion, activation and interaction with the vascular endothelium and infiltration of leukocytes into the brain.

### CROSS REFERENCE

This application claims benefit of U.S. Provisional Patent Application No. 62/334,986, filed May 11, 2016, which application is incorporated herein by reference in its entirety.

### FIELD OF THE INVENTION

The present invention relates to the field of neurology and pharmacology. More specifically, the present invention describes a method for the prevention and treatment of Alzheimer's disease, mild cognitive impairment and other brain inflammatory and neurodegenerative disease by inhibition of T cell immunoglobulin and mucin domain protein-1 (TIM-1) and/or P-selectin function. The discoveries described show that blockade of the TIM-1 or P-selectin prevents and/or reduces cognitive decline in Alzheimer's disease and thus constitutes a therapeutic approach to treat and/or prevent Alzheimer's disease.

### BACKGROUND OF THE INVENTION

T-cell immunoglobulin and mucin domain-1 (TIM-1), also known as KIM-1 and hepatitis A virus cellular receptor 1 (HAVcr-1), is protein encoded by the HAVCR1 gene and is a member of the TIM family, which plays critical roles in regulating immune cell activity and viral infection. The TIM gene family encodes a class of transmembrane glycoproteins expressed by several subsets of immune cells (Kuchroo et al., Nat. Rev Immunol. 8(8): 557-80, 2008). TIM proteins play a role in some immune system functions and are involved in several inflammatory pathologies, including atopic and autoimmune diseases (Rodriguex-Manzanet R., et al., 2009, Immunol. Rev., 229: 259-270 and Freeman G J, et al., 2010, Immunol. Rev, 235: 172-189). Eight TIM members were postulated in mice, encoded by Tim genes located in chromosomal region 11B1.1. By contrast, only three TIM genes were found in humans (encoding TIM-1, TIM-3, and TIM-4) in chromosomal region 5q33.2, which is associated with asthma, allergy, and autoimmunity (Angiari and Constantin 2014 Trends in Molec. Med 20:675-684, and Freeman G J, et al., 2010, Immunol. Rev, 235: 172-189). In both humans and mice, TIM proteins share common structural motifs, including an immunoglobulin V (IgV)-like region in the extracellular region followed by a mucin-like domain) Freeman G J, et al., 2010, Immunol. Rev, 235: 172-189). The TIM protein IgV-like domain has functional similarities to C-type lectins and proteins from the sialic acid-binding immunoglobulin-type lectin (SIGLEC) family (Wilker P R, 2007, Int. Immunol, 19: 762-773) whereas the mucin-like domain contains predicted sites for O-linked and N-linked glycosylation, whose number varies among the different TIM proteins. The single transmembrane region of TIM proteins is followed by a cytoplasmic tail that usually contains tyrosine phosphorylation motifs involved in transmembrane signaling, although TIM-4 is an exception.

### TIM-1 Ligands and Function

TIM-1 was initially identified as hepatitis A virus (HAV) cellular receptor 1 (HAVCR-1), and is necessary for virus uncoating and infectivity. The ligands for TIM-1 were shown to include phosphatidylserine (PS; present in the viral envelope and on the surface of apoptotic cells; reviewed in Moller-Tank and Maury, Virology 468-70, 565-580, 2014) and IgA, as well as Ebola virus glycoproteins. TIM-1 was also identified as kidney injury molecule 1 (KIM-1; Bonventre 2014, Trans Am Clin Climatol Assoc 125; 293-9). It is upregulated and released by renal epithelial cells following kidney injury. In this context KIM-1 is thought to mediate uptake of apoptotic cells, via PS, as well as oxidized LDL into the proximal tubule.

Subsequently, TIM-1 was found to play an important role in immune system activation. TIM-1 expression has been reported on activated CD4 but not CD8 T cells, and TIM-1 cross-linking on naïve T cells leads to rapid T cell activation independent of T cell receptor (TCR) signaling, even though TIM-1 is also recruited to the TCR signaling complex and sustains T cell activation. TIM-1 is mainly thought to acts as a co-stimulatory molecule for T cells following TCR cross-linking, particularly after Th2 polarization, rather than in the direct control of T cell activation. TIM-1 crosslinking also increases Th1, Th17, and Th2 responses in vivo, whereas blocking TIM-1 induces the generation of Treg cells and allograft survival in transplant models.

TIM-1 is also a marker of murine regulatory B cells, and is expressed by activated B cells where it regulates maturation to plasma cells and antibody production. TIM-1 has been detected on DCs, where it confers pro-inflammatory properties, and on mast cells, where it controls Th2-type cytokine production. TIM-1 also serves as a pattern recognition receptor on invariant natural killer cells (iNKT), mediating cell activation when the iNKT cells bind to PS on the surface of cells undergoing apoptosis. Furthermore, recent reports indicate that TIM-1 is a receptor for Zaire Ebola virus and Lake Victoria Marburg virus on mucosal cells, and targets intracellular proteins for degradation. Interestingly, together with TIM-3 and TIM-4, TIM-1 was recently shown to inhibit HIV and Ebola virus release from infected cells, highlighting a new mechanism for the control of viral infection by TIM proteins.

Data from animal models clearly demonstrate that TIM-1 is involved in the development of several immune-related diseases. Blocking TIM-1 significantly reduced airway inflammation and allergic asthma in mouse models, confirming the role of TIM-1 in atopic-like pathologies. Similarly, anti-TIM-1 antibodies reduced ischemia-reperfusion injury in animal models and ameliorated inflammation-associated damage in mouse models of systemic lupus erythematous (SLE) and experimental glomerulonephritis. Interfering with TIM-1-mediated immune processes also severely curtailed the development of skin hypersensitivity and experimental autoimmune encephalomyelitis (EAE), the mouse model of human multiple sclerosis (MS). Together with the involvement of TIM-1 in transplant tolerance, these data clearly indicate that TIM-1 is involved in immune responses and immune-related pathologies (reviewed in Angiari S and Constantin G, 2014, Trends in Molecular Medicine, 20:675-684).

We recently showed that TIM-1 glycoprotein is a ligand for endothelial selectins and that it controls the tethering and rolling of activated T cells in the inflamed microcirculation and the accumulation of T cells at inflammation sites (Angiari S et al., 2014, Immunity 40(4):542-553). PSGL-1, the well-known P-selectin ligand, has been shown to mediate rolling of neutrophils, monocytes and T cells; however, we showed that TIM-1 plays a more specialized role in the trafficking of T cells distinct from PSGL-1. PSGL-1 appears to be involved in naïve T cell homing to lymphoid organs and leukocyte trafficking during inflammation whereas TIM-1 has a specialized role in activated T cell recruitment to sites of inflammation, suggesting a level of diversity between PSGL-1 and TIM-1. We also showed that TIM-1 mediates T cell trafficking in three models of inflammatory conditions: thrombin-activated mesenteric vessels, the inflamed brain endothelium, and in contact hypersensitivity models in the skin, in which P-selectin on the skin endothelium is necessary for activated T cell recruitment. Together these data suggest that TIM-1 is important to achieve tissue specificity in T lymphocyte trafficking. We also showed that TIM-1 is required for the recruitment of Th1 and Th17 cells, potent inducers of inflammation and autoimmunity, suggesting that interference with TIM-1 activity might provide a therapeutic approach in T cell mediated diseases (Angiari S et al., 2014, Immunity 40(4):542-553). Together our findings showed that TIM-1 is a major P-selectin ligand and a pivotal trafficking mechanism for Th1 and Th17 cells during inflammation and show that primary adhesion of T cells to P-selectin in vivo is not exclusively dependent on PSGL-1, but also requires TIM-1, thereby providing a form of concurrency in T cell trafficking between these two critical components of the immune system.

### Diagnosis, Stages, Treatment and Biomarkers for Alzheimer's Disease: Unmet Medical Need

Alzheimer's disease (AD) is the most common form of disabling cognitive impairment in the elderly population. The increase in life expectancy of the population and the lack of effective treatments for AD continue to lead to a rapid increase in patients with AD, which represents an untenable burden on the world population. AD is reported to be the sixth leading cause of death in the US, more than 5.2 million Americans are living with the disease, 1 in 3 senior citizens dies with AD or other dementia, AD will cost the US ˜$203 billion, and costs are expected to rise to $1.2 trillion by 2050. AD is the only cause of death among the top 10 causes in America without a way to prevent, cure or even slow its progression.

There are 7 stages during the course of human disease. Stage 1 is characterized by normal cognitive function. Stage 2 is characterized by very mild cognitive decline and the patient may have memory lapses, but no symptoms of dementia can be detected. Stage 3 is mild cognitive decline and is considered an early-stage AD. During a detailed medical interview, doctors may be able to detect problems in memory or concentration. Common difficulties during stage 3 include: problems with the right word or name, trouble remembering names when introduced to new people, noticeable difficulty performing complex tasks, loosing or misplacing objects, increasing trouble with planning or organizing. Stage 4 is moderate cognitive decline and is equivalent of mild or early-stage Alzheimer's disease. During stage 4 the symptoms are forgetfulness of recent events, impaired ability to perform challenging mental arithmetic, greater difficulty performing complex tasks, forgetfulness about one's own personal history, becoming moody or withdrawn, especially in socially or mentally challenging situations. Stage 5 is moderately severe cognitive decline and is considered a moderate or mid-stage AD. During this stage patients present noticeable gaps in memory and thinking and start to need help with day-to-day activities: they are unable to recall their own address, telephone number, become confused about where they are or what day it is, have trouble with simple mental arithmetic. Stage 6 includes severe cognitive decline and is considered a moderately severe or mid-stage AD; memory continues to worsen in these patients, personality changes may take place and individuals need extensive help with daily activities. Stage 7 is a very severe disease and is considered the severe or late-stage AD. In this final stage of disease, individuals lose the ability to respond to their environment, to carry a conversation and, eventually, to control movement.

The clinical progressive decline of cognitive and behavioral symptoms is concordant with neuronal loss, synaptic dysfunction and atrophy in brain regions linked to learning and memory. Two pathophysiological hallmarks of AD are well characterized: accumulation of amyloid-beta (Aβ) peptide into amyloid plaques in the extracellular brain parenchyma and the formation of tangles inside neurons as a result of abnormal phosphorylation of the microtubule-associated protein tau. Amyloid deposits and tangles are accompanied by a marked loss of neurons in the neocortex and hippocampus.

AD is generally diagnosed using behavioral and neurophysiological tests, such as the mini-mental state examination (MMSE). Patients are tracked using neurophysiological tests to measure cognition, memory and social functioning. Biochemical and neuroimaging biomarkers are also used to track disease status, including positron emission tomography (PET) using ligands that detect, for example, beta amyloid or phosphorylated tau, and magnetic resonance imaging (MRI) measurements of hippocampal atrophy. Biomarkers in the cerebrospinal fluid can also be monitored, including for example, phosphorylated tau, A-beta, and synaptic biomarkers such as neurogranin, which reflect key aspects of disease pathogenesis, such as neuronal degeneration, phosphorylation of tau with tangle formation, and the aggregation and deposition of A-beta into plaques (Lleo et al 2015 Nature Reviews Neurology 11, 41-55 and references therein).

AD is ultimately fatal. Death generally occurs within 3 to 9 years after diagnosis. There is no cure. There is no effective treatment for prevention, treatment or slowing of decline for AD patients. The U.S. Food and Drug Administration have approved five drugs that temporarily improve symptoms. The effectiveness of these drugs varies across the population, but at best they are only moderately effective in stabilizing or improving cognitive and functional symptoms for 6-12 months. None of the treatments available today alters the underlying course of this terminal disease. Clearly there is an urgent unmet medical need for effective therapeutics and approaches to treat AD. The present invention addresses this need, and provides surprising benefits of blocking neutrophil and/or myeloid cell activation, adhesion and/or invasion of the brain to treat Alzheimer's and other neurodegenerative disease.

### BRIEF SUMMARY OF THE INVENTION

Methods are provided for the prevention and treatment of neurological and/or neurodegenerative disease in an individual, including without limitation treatment of an individual with Alzheimer's disease (AD) and mild cognitive impairment (MCI). In the methods of the invention, an individual suffering from, or pre-disposed to, a neurological disease is contacted with an effective dose of an agent that blocks the P-selectin pathway or the TIM-1-pathway to prevent, reduce or reverse cognitive decline and other symptoms of neurodegeneration. An effective dose of an agent of the invention can be administered before or well after symptoms are evident and can be administered continuously or intermittently and may be used in combination with other therapeutic agents.

Agents of the invention target TIM-1, P-selectin, the TIM-1 and/or P-selectin adhesion pathways. In some embodiments an agent of the invention binds to and inhibits the activity of TIM-1. In some embodiments an agent of the invention binds to and inhibits P-selectin. In some embodiments an agent of the invention binds to and inhibits the activity of PSGL-1. In some embodiments an agent of the invention binds to and inhibits the activity of LFA-1. In some embodiments a combination of agents is administered, sequentially or concomitantly, comprising an agent that inhibits TIM-1 pathway and an agent that inhibits P-selectin pathway. In some such embodiments an agent that inhibits TIM-1 is administered in combination with an agent that inhibits PSGL-1.

In some embodiments an agent is an antibody or active fragment or derivative thereof. In some embodiments an agent is a small molecule. In some embodiments an agent is a peptide or peptidomimetic. In some embodiments the agent is a glycan or glycomimetic.

An effective dose of an agent is provided for a period of time sufficient to reduce the presence, adhesion, activation or function of immune or vascular cells or platelets and block leukocyte-vascular, or leukocyte-platelet, leukocyte-leukocyte, vascular-platelet or leukocyte fragment interactions or leukocyte activation in a targeted region of the brain, which region may comprise, without limitation, the vasculature or the parenchyma of the brain and/or at the site of neurodegenerative or neurological lesions, e.g. at plaques associated with AD.

In some embodiments the agent is not required to cross the blood brain barrier. In some embodiments the agent is administered systemically.

### DETAILED DESCRIPTION OF THE INVENTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events that is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The methods of the invention find use in a wide variety of animal species, particularly including mammalian species. Animal models, particularly small mammals, e.g. murine, lagomorpha, etc. are of interest for experimental investigations. Other animal species may include, e.g. horses, cattle, rare zoo animals such as panda bears, large cats, etc. Humans are of particular interest. Individuals of interest for treatment with the methods of the invention include, without limitation, those suffering from Alzheimer's disease.

### Definitions

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

PSGL-1. P-selectin glycoprotein ligand-1 (PSGL-1), also known as SELPLG or CD162, is a high affinity ligand for P-selectin on myeloid cells and stimulated T cells. It plays a critical role for tethering of cells to activated platelets or endothelial cells expressing P-selectin. Selectin binding to PSGL-1 requires both sulfation of tyrosines and the addition of sialyl Lewis^{x} tetrasaccharide (sLE^{x}) to O-linked glycans on PSGL-1.

SLex. Sialyl-Lewis^{X} (Sle^{X}) is a tetrasaccharide carbohydrate attached to O-linked glycans (carbohydrates linked to serine or threonine residues on a peptide). The presence of a proline residue at −1 or +3 relative to the serine or threonine is favorable for O-linked glycosylation. O-linked glycans that are capped with a sialic acid residue with a penultimate fucose forms the sLex structure.

Alzheimer's disease. Alzheimer's disease is a progressive, inexorable loss of cognitive function associated with an excessive number of senile plaques in the cerebral cortex and subcortical gray matter, which also contains β-amyloid and neurofibrillary tangles consisting of tau protein. The common form affects persons >60 yr old, and its incidence increases as age advances. It accounts for more than 65% of the dementias in the elderly.

The cause of Alzheimer's disease is not known. The disease runs in families in about 15 to 20% of cases. The remaining, so-called sporadic cases have some genetic determinants. The disease has an autosomal dominant genetic pattern in most early-onset and some late-onset cases but a variable late-life penetrance. Environmental factors are the focus of active investigation.

In the course of the disease, neurons are lost within the cerebral cortex, hippocampus, and subcortical structures (including selective cell loss in the nucleus basalis of Meynert), locus caeruleus, and nucleus raphae dorsalis. Cerebral glucose use and perfusion is reduced in some areas of the brain (parietal lobe and temporal cortices in early-stage disease, prefrontal cortex in late-stage disease). Neuritic or senile plaques (composed of neurites, astrocytes, and glial cells around an amyloid core) and neurofibrillary tangles (composed of paired helical filaments) play a role in the pathogenesis of Alzheimer's disease. Senile plaques and neurofibrillary tangles occur with normal aging, but they are much more prevalent in persons with Alzheimer's disease.

The essential features of dementia are impairment of short-term memory and long-term memory, abstract thinking, and judgment; other disturbances of higher cortical function; and personality change. Progression of cognitive impairment confirms the diagnosis, and patients with Alzheimer's disease do not improve.

AD animal models. In 2011 the National Institute on Aging and Alzheimer's Association (NIA-AA) proposed a new framework for characterizing preclinical AD in man. Stage 1 includes abnormal levels of Aβ; stage 2 includes abnormal levels of Aβ and evidence of brain injury; stage 3 abnormal levels of Aβ and evidence of brain injury plus subtle cognitive changes. The clinical definitions of stages 1-6 are described above.

Based on the new definition from the NIA-AA and the clinical stages described in the introduction/background, and the time-course of disease in the mouse models, the timing of the therapeutic interventions described in the mouse models are defined as early clinical stage 3 to 4 and preclinical stage 4 and are defined as early to mid-stage of AD.

It has been shown that patients with familial AD (FAD) present mutations of genes encoding APP itself or protease subunits such as presenilin (PS) 1 and 2 involved in APP cleavage to generate Aβ. The discovery of mutated APP and PS was the basis for generation of transgenic animal models harboring these human mutations and thus closely replicating cardinal features of AD. Transgenic mouse models have greatly advanced the understanding of AD pathogenesis. Transgenic mice overproducing human APP containing familial AD mutations show increased production of Aβ, which accumulates with age into diffuse or compact amyloid plaques. The mice show synaptic transmission deficits that often precede the formation of the plaques. Overexpression of presenilin1 further increases Aβ production and accelerates pathology. Mice overexpressing human tau protein mutants that are associated with familial forms of frontotemporal dementia and Parkinsonism linked to chromosome 17—a dementia characterized by extensive tangle formation develop neurofibrillary tangles similar to those observed in AD. Mice expressing the P301 mutant tau mimic features of human tauopathies.

A mouse model has been described, the 3xTG model, that harbors all three mutant genes, tauP^{301L}, APP^{K670N, M671L} and PS1^{M146V}. 3xTg-AD mice produces amyloid plaques and tangles, shows synaptic transmission defects, and develop age-related and progressive neuropathological phenotype in the hippocampus amygdala and cerebral cortex, the most pronounced brains structures impacted by AD pathology. Intracellular Aβ is apparent between 3 and 4 months of age in the neocortex and by 6 months in the hippocampus. Neurofibrillary alterations tau pathology (hyperphosphorylated and/or conformationally altered tau) is observed between 12 and 15 months of age. Tau-reactive dystrophic neuritis is evident in older 3xTg-AD brains (18 mo). Of note, Aβ and tau pathology initiate in different brain regions in 3xTg-AD mice (i.e. cortex for Aβ and hippocampus for tau). This is not inconsistent with the notion that Aβ influences tau pathology, but suggests that a soluble intracellular Aβ, other soluble factors and/or motile cells are involved in Aβ-mediated tau pathology. By 6 months (the earliest time points tested after baseline measurements at 1 month) synaptic dysfunction and long-term potentiation (LTP), which is involved in learning and memory) was severely impaired in 3xTg-AD mice compared to wild-type-aged matched mice (Oddo et al., 2003. Neuron: 39(3):409-21).

Neurodegenerative disease. Neurodegenerative disease involves the progressive loss of the structure and function of neurons or brain structures including, but not limited to, Alzheimer's disease, dementia, mild cognitive impairment, amylotrophic lateral sclerosis (ALS), Parkinson's, Huntington's, vascular dementia, other forms of dementia, drug and diabetes-induced neurodegeneration, epilepsy, head trauma, tramautic brain injury and others.

Leukocyte trafficking and endothelial cell interactions. The endothelial interface/endothelium produces a large number of soluble factors that can influence systemic and local tissue function. Once bound to the endothelium or infiltrated into the tissue, leukocyte subpopulations such as neutrophils can damage tissue through release of ROS, and proteases and drive inflammation via secretion of cytokines, chemokines, and leukotrienes.

Leukocyte recruitment is the primu movens of any immune response and is critical to the onset of inflammatory and autoimmune disease. The molecular mechanisms involved in leukocyte-endothelial cell adhesive interactions have been extensively reviewed (Ley K et al., 2007, Nat Rev Immunol. 7:678-689 and references therein). Briefly, leukocyte recruitment is a complex process controlled by both molecular and mechanochemical events. Leukocytes circulating in the blood are selectively recruited to specific target sites through multi-step cascades of adhesive interactions and activating signals. Several specialized receptors of the selectin, mucins and integrin super gene families are expressed by leukocytes and have evolved to establish shear-resistant adhesions within seconds. The initial tethering and rolling are mainly mediated by selectins (although also VLA-4 and LFA-1 may mediate rolling under specific circumstances. In contrast, rapid stable adhesion (arrest) on vascular endothelium is mediated, depending on the leukocyte subset and endothelial ligand available, by cooperation between integrins, including α₄ integrins such as VLA-4 (α₄β₁, CD49d/CD29) or α₄β₇ and the β₂ integrins LFA-1 (αLβ₂, CD11a/CD18) and Mac-1 (αMβ₂, CD11b/CD18). These integrins recognize counter receptors expressed on the endothelium and belonging to the immunoglobulin supergene family, such as ICAM-1 (CD54), VCAM-1 (CD106) and MAdCAM-1. Circulating leukocytes maintain integrins largely in non-adhesive state. However, once integrins have been properly activated in situ, leukocytes stably adhere to the endothelial cells. The most powerful physiologic integrin activators are classical chemoattractants, such as LTB4, PAF, C5a and fMLP, and chemotactic cytokines (chemokines) such as CXCL8, CXCL12, CCL21, CCL19, CCL17, CCL22 and many others which trigger intracellular signaling networks leading to a very rapid integrin activation and subsequent stable adhesion. Once arrested on the endothelium the journey of the leukocyte is not over. The leukocyte needs to complete the process by spreading and crawling on the surface and transmigrates, by transcellular or paracellular routes, through the endothelium to definitively extravasate and enter into the tissue, a process called diapedesis. Also this phenomenon relies on integrin activation and is facilitated by the flow.

Neutrophil tethering and rolling are mediated by selectins; L-selectin is expressed constitutively on neutrophils, whereas activated endothelial cells express E- and P-selectins. The selectins interact with their counter-receptors on leukocytes and endothelial cells. Recruitment of neutrophils requires CD11/CD18 complexes under most circumstances; however, non-CD11/CD18-mediated neutrophil emigration has been demonstrated certain pathological conditions in CD18 deficient mice. Neutrophils are generally assumed not to express alpha-4 integrin; however, neutrophils have been shown to express alpha 4 integrin under certain conditions in vitro and in vivo and the alpha4-integrin-VCAM-1 pathway for neutrophil recruitment has been demonstrated in human disease. The alpha-4 pathway can mediate tethering, rolling and adhesion under flow conditions on VCAM-1 and MAdCAM-1.

In vitro and in vivo studies have established that leukocyte arrest is rapidly triggered by chemokines or other chemoattractants and is mediated by the binding of leukocyte integrins to immunoglobulin superfamily members, such as ICAM1 and VCAM1, expressed by endothelial cells. LFA-1 integrin is one of the most relevant to leukocyte arrest and classical chemoattractants are the most powerful physiological activators of LFA-1-mediated adhesion in vivo. Ligation of specific hetero-trimeric GPCRs by chemokines activate integrins by triggering a complex intracellular signaling network within milliseconds leading to conformational changes leading to increased affinity, and lateral mobility leading to increased valency, the increase of both integrin affinity and valency, both enhancing cell avidity (adhesiveness).

Inside-out signaling induces integrins to undergo a dramatic transition from a bent low-affinity conformation to extended intermediate- and high-affinity conformations, which leads to opening of the ligand-binding pocket.

In this context, integrin activation is a key step since it mediates rolling (in certain conditions), arrest and diapedesis of activated leukocytes. The most potent agonists for integrin triggering are chemotactic factors, such as formylated peptides or chemokines. Chemotactic factors receptors trigger a very complex intracellular signaling network leading to various kinetic aspects of integrin-mediated adhesion. Overall, at least 65 signaling proteins are involved in the regulation of integrin-mediated adhesion by chemoattractants.

The selectins mediate adhesion of hematopoietic cells, including neutrophils and myeloid cells, to vascular cells and to each other. These interactions are key for host defense, immune cell surveillance and inflammation. Reversible interactions with E- and P-selectin expressed on endothelial cells mediate tethering and rolling in inflamed vascular beds. The selectins are calcium-dependent lectins that bind to glycan determinants on a variety of proteins and lipids. There are a large number of selectin ligands, including PSGL-1, CD44, E-selectin ligand, and CD43 that can play a role in myeloid cell/neutrophil interaction with the vasculature. Some of these ligands can mediate signaling cascades, for example PSGL-1 and CD44 induce signals that activate beta-2 integrin LFA-1 and ESL-1 can activate the Beta2 integrin MAC-1 in neutrophils. TIM-1 is expressed on activated T cells and mediates T cell trafficking under inflammatory conditions.

PSGL-1 is a major selectin ligand on leukocytes. PSGL-1 is the predominant ligand for P-selectin, but it can also bind to E- and L-selectin under flow conditions and mediates leukocyte tethering and rolling, and can transduce signals into rolling leukocytes and into leukocytes decorated with platelets.

Leukocyte Activation. Tyrosine kinases (PTK) are of interest for targeting leukocyte activation. PTKs are normally upstream transducers in signaling cascades, thus they may behave as massive amplifiers and regulators of signaling events. Signaling through PSGL-1, the main selectin receptor on activated T cells, depends on constitutive binding between the PSGL-1 cytoplasmic tail and Nef-associated factor 1 (Naf1; Wang, H. B. et al. 2007 Nat. Immunol. 8, 882-892). The binding of P-selectin to PSGL-1 leads to the phosphorylation of Naf1 by Rous sarcoma (Src) family kinases, and subsequent recruitment of the phosphatidylinositol-4,5-bisphosphate 3-kinase (PI3K) p85-p1108 heterodimer, which triggers the activation of β₂ integrins (Wang, H. B. et al. 2007 Nat. Immunol. 8, 882-892). As shown for PSGL-1, the TIM-1 intracellular tail is phosphorylated by Src family kinases (de Soiusa et al., J Immunol., 2008. 180, 6518-6526), and TIM-1 crosslinking induces the phosphorylation of its cytoplasmic tail as well as Zap-70 and IL-2-inducible T-cell kinase (ITK) (Binné et al., J Immunol. 2007]. Interestingly, the p85 subunit of PI3K is recruited directly to the tyrosine-276 residue of TIM-1 after phosphorylation of the cytoplasmic tail (de Sousa et al. 2008 J. Immunol. 178, 4342-4350). Thus, TIM-1 may be involved in integrin transactivation via PTK activation.

As used herein, an “antagonist,” refers to a molecule which, when interacting with (e.g., binding to) a target protein, decreases the amount or the duration of the effect of the biological activity of the target protein (e.g., interaction between leukocyte and endothelial cell in recruitment and trafficking). Antagonists may include proteins, protein fragments, nucleic acids, carbohydrates, antibodies, anti-sense nucleotides or any other molecules that decrease the effect of a protein. Unless otherwise specified, the term “antagonist” can be used interchangeably with “inhibitor” or “blocker”.

The term “agent” as used herein includes any substance, molecule, element, compound, entity, or a combination thereof. It includes, but is not limited to, e.g., protein, oligopeptide, small organic molecule, polysaccharide, polynucleotide, anti-sense nucleotides, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a molecule of interest but which has been modified in a targeted and controlled manner, by replacing a specific substituent of the reference molecule with an alternate substituent. Compared to the starting molecule, an analog may exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry.

Antagonists of interest include antibodies specific for one or more adhesion molecules involved in leukocyte recruitment or trafficking to the central nervous system. Also included are soluble receptors, conjugates of receptors and Fc regions, and the like. Generally, as the term is utilized in the specification, “antibody” or “antibody moiety” is intended to include any polypeptide chain-containing molecular structure that has a specific shape which fits to and recognizes an epitope, where one or more non-covalent binding interactions stabilize the complex between the molecular structure and the epitope. The archetypal antibody molecule is the immunoglobulin, and all types of immunoglobulins (IgG, IgM, IgA, IgE, IgD, etc.), from all sources (e.g., human, rodent, rabbit, cow, sheep, pig, dog, other mammal, chicken, turkey, emu, other avians, etc.) are considered to be “antibodies.” Antibodies utilized in the present invention may be polyclonal antibodies, although monoclonal antibodies are preferred because they may be reproduced by cell culture or recombinant methods, and may be modified to reduce their antigenicity.

Antibody fusion proteins may include one or more constant region domains, e.g. a soluble receptor-immunoglobulin chimera, refers to a chimeric molecule that combines a portion of the soluble adhesion molecule counter-receptor with an immunoglobulin sequence. The immunoglobulin sequence preferably, but not necessarily, is an immunoglobulin constant domain. The immunoglobulin moiety may be obtained from IgG1, IgG2, IgG3 or IgG4 subtypes, IgA, IgE, IgD or IgM, but preferably IgG1 or IgG3.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

Small molecule agents encompass numerous chemical classes, though typically they are organic molecules, e.g. small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides and anti-sense nucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of GABA inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

Candidate antagonists can be tested for activity by any suitable standard means. As a first screen, the antibodies may be tested for, binding against the activation molecule, adhesion molecule, etc. As a second screen, candidates may be tested for binding to an appropriate cell line, e.g. leukocytes or endothelial cells, or to primary tissue samples. For these screens, the candidate may be labeled for detection (e.g., with fluorescein or another fluorescent moiety, or with an enzyme such as horseradish peroxidase). After selective binding to the target is established, the candidate produced as described below, may be tested for appropriate activity, including the ability to block leukocyte recruitment to the central nervous system in an in vivo model, such as an appropriate mouse or rat epilepsy model, as described herein.

Currently available therapeutic agents for blocking leukocyte recruitment include polypeptide therapeutics, e.g. antibodies, monoclonal antibodies, anti-sense nucleotides receptor-Fc chimeric fusion proteins, etc., and small molecule-based drugs. There are now multiple clinically validated anti-adhesion drugs. Small-molecule antagonists of adhesion molecule function can be categorized into three distinctive modes of action: ligand-mimetic competitive antagonists and allosteric antagonists or a I allosteric antagonists.

Approved therapies comprise an antibody fragment, ReoPro™, and two small-molecule inhibitors, Integrilin™ and Aggrastat™. These structures built on previously published structures of an integrin binding to its RGD based ligand. This information may yield additional routes to drug discovery that target medically relevant integrins.

Advances in the functional understanding of carbohydrate-protein interactions have enabled the development of a relatively new class of small-molecule drugs, known as glycomimetics. These compounds mimic the bioactive function of carbohydrates and address the drawbacks of carbohydrate leads, namely their low activity and insufficient drug-like properties. Examples of glycomimetic, small-molecule antagonists of the P-selectin or all three known selectins, are: Cylexin (CY-1503) from Cytel, Bimosiamose (TBC-1269) from Revotar, OJ-R9188 from Nippon Organon, GM1070 from Glycomimetics, PSI-697 from Wyeth, GSC-150 from Kanebo and Efomycin M from Bayer (Ernst and Magnani, Nat Revs Drug Discov 2009). An anti-human P-selectin antibody (SeIG1) from Selexys Pharmaceuticals is currently under investigation (SUSTAIN study) for its potential to reduce or prevent the occurrence of sickle cell-related pain crises.

“Target acquisition”, as used herein, refers to the successful interaction of a drug or biologic agent with the target cell or molecule. Target acquisition may be monitored by methods suitable for the specific interaction, e.g. a flow cytometry assay to look at saturation levels of an antibody on a target cell, calcium flux in a target population for a signaling molecule; and the like.

### Methods of the Invention

Methods are provided for the prevention and treatment of neurological and/or neurodegenerative disease in an individual, including without limitation Alzheimer's disease (AD) and mild cognitive impairment (MCI). The methods of the invention are based, in part, on the discovery that blockade of TIM-1, a newly identified ligand of P-selectin, reduces cognitive impairment, beta-amyloid deposition, presence of phosphorylated tau, and the number and activation of microglia and in a model of AD. The methods are also based, in part, on the discovery that blockade of P-selectin also reduces cognitive impairment in a model of AD.

In a preferred embodiment of the invention, an individual suffering from, or pre-disposed to, a neurological disease or neurodegenerative disease, including but not limited to AD or MCI, is contacted with an effective dose of an agent that blocks the TIM-1 and/or P-selectin adhesion pathway activity for a period of time sufficient to prevent, reduce or reverse cognitive decline and other symptoms of neurodegeneration, measured using standard clinical assessment protocols and methods.

The methods of the invention are based in part on the discovery that mid-to-late stage (initiated at 9 months when cognitive decline is clearly evident), short-term blockade of P-selectin and/or TIM-1 results in protection against cognitive decline measured after a 1 month wash-out period. As used herein, short term may refer to a period of up to about 1 week, up to about 2 weeks, up to about 3 weeks, up to about 4 weeks, up to about 5 weeks. Protection may also be obtained by longer term administration of the agent, e.g. for a period of up to about 1 month, up to about 2 months, up to about 3 months, up to about 4 months, up to about 6 months or longer, where dosing can be continuous or intermittent, e.g. administering the agent 1 day a week, 2 days a week, 3 days a week, 4 days a week; 5 days a week, 6 days a week, every day in a week; every week, every other week, every third week; etc.

An effective dose of an agent of the invention can be administered before or well after symptoms are evident and can be administered continuously or intermittently and may be used in combination with other therapeutic agents. Using ordinary skill, the competent clinician will be able to select and track patients and optimize the dosage and regimen of a particular therapeutic using standard behavioral and neurophysiological tests, such as the mini-mental state examination (MMSE), to measure cognition, memory and social functioning in the course of routine clinical trials.

The methods are also based on the finding that biomarkers, including A-beta and phosphorylated tau, are reduced by blockade of TIM-1 in a model of AD. The methods are also based on the finding that the number and activation status of microglial cells are reduced by blockade of TIM-1 in a model of AD. The effective dose and regimen can be monitored and determined by a competent clinician in the course of routine clinical using standard biochemical and neuroimaging biomarkers to track disease status and progression, including positron emission tomography (PET) using ligands that detect, for example, beta amyloid or phosphorylated tau, and magnetic resonance imaging (MRI) measurements of hippocampal atrophy. Biomarkers in the cerebrospinal fluid can also be monitored, including phosphorylated tau, A-beta, and synaptic biomarkers such as neurogranin or phosphotagamin, which reflect key aspects of disease pathogenesis, such as neuronal degeneration, phosphorylation of tau with tangle formation, the aggregation and deposition of A-beta into plaques, and the number and activation status of microglia using standard methodology known to competent clinicians in the field (Lleo et al, 2015. Nat Rev Neurol. 11(1):41-55 and references therein).

In some embodiments, an effective dose of an agent targeting one or both of TIM-1 or P-selectin is provided for a period of time sufficient to impact the adhesiveness, activation, adhesion and/or recruitment of leukocytes, myeloid cells and/or neutrophils and/or platelets in the in the region of the brain, which region may include the vasculature of the brain; which may include a reduction of interactions with the brain vasculature and/or at the site of neurodegenerative or neurological lesions, e.g. at plaques associated with AD. The effective dose and regimen to sufficiently block target cell activity can be monitored standard methods known to those skilled in the art, to assess, for example, the number and/or ratio of the target cells to other cells in the blood, binding saturation of the therapeutic on the cell, target cell activation status, adhesiveness, integrin affinity status, adhesion molecule clustering or similar, on the circulating leukocytes from a treated patient.

In some embodiments neurodegenerative disorders that can be treated with the methods of the invention include, without limitation, ALS, Parkinson's disease, Huntington disease, diabetes-induced neurodegeneration, epilepsy, stroke, head trauma, vascular dementia and other forms of dementia. Individuals suffering from or at risk of developing a neurodegenerative disorder is treated with an effective dose or dosing regimen of a therapeutic agent targeting one or both of the TIM-1 and P-selectin pathways capable of reducing or reversing cognitive decline.

It is shown herein that TIM-1 is involved in multiple steps in leukocyte-vascular interactions, including capture, rolling, arrest, activation and spreading of neutrophils under flow conditions. TIM-1-mediated neutrophil adhesion involves the IgV and mucin domains of TIM-1. It is further shown herein that both PSGL-1 and LFA-1 contribute to TIM-1-mediated neutrophil adhesion.

Agents of the invention include, but are not limited to, those that target the IgV domain of TIM-1, the mucin domain of TIM-1, the sialic acid binding site of TIM-1, the TIM-1 binding site of PSGL1 or LFA-1, the PSGL-1 binding site of TIM-1, the P-selectin binding site of TIM-1, PSGL1 or LFA-1, the LFA-1 binding site of TIM-1, PSGL-1 or P-selectin and/or domains and sites involved in integrin activation. Agents of the invention include but are not limited to those that block TIM-1-mediated stop-and-go, tethering, rolling, rapid adhesion, leukocyte activation, integrin activation, spreading and/or diapedesis of leukocytes. The selection and effectiveness of agents can be assessed using the assays described in the examples and similar assays standard in the art.

One embodiment of the invention provides for administration of an agent targeting P-selectin on vascular endothelial cells, platelets, leukocytes or cell fragments within the vasculature. In this embodiment the agent is not required to cross the BBB.

One embodiment of the invention provides for administration of an agent targeting TIM-1 on cells including, without limitation, endothelial cells, leukocytes, and/or platelets and cell fragments attached on the endothelial cells and/or platelets, which modulate leukocyte-vascular interactions (LVI) or leukocyte activation. In this embodiment the agent is not required to cross the BBB.

One embodiment of the invention provides for administration of an agent targeting TIM-1 in CNS parenchyma, for example TIM-1 on leukocytes or other cells, including neural cells, or cell fragments. In this embodiment can be administered systemically or locally, for example via intrathecal or intranasal administration.

In some embodiments of the invention the therapeutic agent blocks the interactions between leukocytes, endothelial cells and/or platelets and/or activation of leukocytes, endothelial cells and/or platelets, blocking TIM-1 function resulting in blockade of leukocyte activation, for example, but not limited to, blockade of triggering of integrin LFA-1 to a high affinity state, as assessed using reagents that recognize the low, medium and high-affinity states of LFA-1 and similar assays standard in the art. In some embodiments of the invention the agents used to inhibit TIM-1 function target the mucin domain and/or carbohydrate recognition and binding of TIM-1, such agents including, but not limited to, glycomimetics or glycosylated inhibitors of the sialic acid binding domain or other carbohydrate binding domains of TIM-1. In other embodiments the agents used to inhibit TIM-1 function target the IgV domain of TIM-1.

Activated platelets can express P-selectin and thus may interact with cells expressing TIM-1. Blockade of TIM-1 can be useful for diseases in which platelet adhesion play a role in disease pathogenesis, e.g. cerebral ischemia, atherosclerosis, coronary artery thrombosis and other cardiovascular diseases, such as syncope, peripheral vascular disease and others related to mellitus diabetes. All these diseases involve vascular occlusion and direct participation of platelets (Geraldo et al 2014 Int J Mol Sci 15(10):17901). The agonists and/or antagonists of the present invention are administered at a dosage that modulates leukocyte-vascular interaction whilst minimizing any side-effects. It is contemplated that compositions will be obtained and used under the guidance of a physician for in vivo use. The dosage of the therapeutic formulation will vary widely, depending upon the nature of the disease, the frequency of administration, the manner of administration, the clearance of the agent from the host, and the like.

Embodiments of the invention include methods to treat or prevent MCI, AD, and related neurodegenerative disease by any single or combination of the following methods (i) blocking neutrophils/myeloid/lymphocyte cell adhesion and crawling; (iii) blocking transmigration and infiltration of neutrophils/myeloid/lymphocyte cells into the brain; (iv) blocking cell-cell interactions between neutrophil/myeloid/lymphocyte cells and endothelial cells and/or neural cells; (v) blocking neutrophil/myeloid/lymphocyte cell extracellular-matrix interactions; (vi) reducing motility of neutrophils/myeloid/lymphocyte cells in the brain parenchyma; (vii) blocking Aβ-induced activation and adhesion of neutrophils/myeloid/lymphocyte cells; (viii) blocking intracellular signaling controlling adhesion and activation; (ix) blocking leukocyte activation and/or degranulation; (x) blocking release of reactive oxygen species, proteases, cytokines, lipid mediators or other damaging agents from myeloid cells and/or neutrophils; (xi) blocking leukocyte activation leading to increased affinity and valency; (xii) blocking formation of neutrophil extracellular traps (NETS) in brain vessels or parenchyma.

Therapeutic agents targeting blockade of P-selectin and PSGL-1 have been developed to target treatment of sickle cell disease. For example, a pan E-, P- and L-selectin glycomimetics as well as anti-P-selectin and anti-TIM-1 MAbs have been developed to treat vasso-occlusive crisis of sickle cell disease. Sickle cell disease is a relatively rare disease afflicting approximately 90,000 to 100,000 US citizens and approximately and 10-15,000 people in the US and France. Patients with sickle cell disease suffer vasooclussive complications in which sickled red blood cells adhere to each other and to platelets and potentially block small blood vessels blocking blood flow resulting in pain crises, and possibly progressive multi-organ dysfunction and premature death. Sickle cells disease is considered an orphan indication by the FDA. This patient population does not generally overlap with the AD patient population.

The patient selection, status, efficacy and target acquisition for agents targeting one or both of P-selectin and TIM-1 in AD are described above and are distinct from those used for sickle cell disease. In sickle cell disease, acute and chronic inflammation can cause endothelial cells and platelets to become activated and P-selectin moves to the surface where it can bind to PSGL-1 on leukocytes and a PSGL-1-like receptor on sickled red cells. P-selectin on activated platelets can also bind to PSGL-1 on leukocytes and endothelial cells. Blockade or elimination of P-selectin has been shown to block these interactions. The efficacy of anti-P-selectin pathway therapy in sickle cell disease would be monitored by examination of erythrocyte shape and function, platelet activation and platelet reactivity, and or erythrocyte-platelet aggregates, the primary cause of the vessel blockade leading to sickle cell crisis.

Therapeutic agents targeting TIM-1 and P-selectin have been developed for treatment of atopic disease including allergic asthma. Asthma is a chronic inflammatory disease of the lung characterized by airflow obstruction and bronchospasm resulting in wheezing, coughing and shortness of breath. Rates of asthma vary between countries with prevalence between 1 and 18%; asthma affects approximately 7% of the US population and is frequently diagnosed among children 10-17 years of age and is more common among the young (Murray and Nadel's textbook of respiratory medicine (5^{1h} edition) Philadelphia, Pa.: Saunders/Elsevier 2010 Chapter 38). This patient population does not generally overlap with the AD population.

Patient selection and tracking for successful blockade of P-selectin for MCI, AD and/or other neurodegenerative disease is distinct from that used for asthma and other atopic disease. Asthma is typically diagnosed based on recurrent episodes of wheezing, breathlessness, chest tightness and coughing as well as by airflow obstruction. Spirometry and other pulmonary function tests, specifically the amount and flow of air that can be inhaled and exhaled, are used to diagnose and monitor patients.

Monitoring and determination of target acquisition following anti-P-selectin/P-selectin blockade or anti-TIM-1 therapy/TIM-1 blockade for MCI and/or AD would be tracked using very different molecular, physiologic, and clinical changes compared to target acquisition of P-selectin and/or TIM-1 in sickle cell disease and asthma. Further, selection of patients P-selectin and/or TIM-1 blockade in prevention and/or treatment of neurodegenerative disease would be based on different molecular, physiologic and clinical changes compared to the selection criteria for therapy in sickle cell disease.

Therapeutic agents targeting TIM-1 for treatment of autoimmune disease, such as multiple sclerosis (MS) and graft rejection have been proposed. MS is the most common autoimmune disorder of the CNS, afflicting 2.5 million people globally. MS generally appears in adults in their late twenties or thirties and this patient population does not generally overlap with the AD population. MS is characterized by T-cell mediated destruction of myelin sheaths of neurons and the loss of oligodendrocytes, the cells responsible for creating and maintaining the myelin sheath, resulting in loss of neuron function and a scar-like plaque around the damaged axons. This process and the methods to track disease progression and efficacy of agents to treat the process are distinct from AD, MCI and related neurodegenerative disease. The patient population and tracking of efficacy for graft-rejection are also distinct from AD and related neurodegenerative disease. In some embodiments of the invention a patient treated with TIM-1 has not been diagnosed with MS.

Therapeutic agents, e.g. agonists or antagonists can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intracheal, etc., administration. The active agent may be systemic after administration or may be localized by the use of regional administration, intramural administration, or use of an implant that acts to retain the active dose at the site of implantation.

One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents is also an option. A BBB disrupting agent can be co-administered with the therapeutic compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture, in vitro binding and flow assays or similar and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, intranasal, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The agents of the invention may be administered using any medically appropriate procedure, e.g. intravascular (intravenous, intraarterial, intracapillary) administration, injection into the cerebrospinal fluid, intracavity or direct injection in the brain. Intrathecal administration may be carried out through the use of an Ommaya reservoir, in accordance with known techniques. (F. Balis et al., Am J. Pediatr. Hematol. Oncol. 11, 74, 76 (1989).

Where the therapeutic agents are locally administered in the brain, one method for administration of the therapeutic compositions of the invention is by deposition into or near the site by any suitable technique, such as by direct injection (aided by stereotaxic positioning of an injection syringe, if necessary) or by placing the tip of an Ommaya reservoir into a cavity, or cyst, for administration. Alternatively, a convection-enhanced delivery catheter may be implanted directly into the site, into a natural or surgically created cyst, or into the normal brain mass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μI/minute), rather than diffusive flow, to deliver the therapeutic composition to the brain and/or tumor mass. Such devices are described in U.S. Pat. No. 5,720,720, incorporated fully herein by reference.

The effective amount of a therapeutic composition to be given to a particular patient will depend on a variety of factors, several of which will be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD₅₀ animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent, e.g. some agents may be taken for extended periods of time on a daily or semi-daily basis, while more selective agents may be administered for more defined time courses, e.g. one, two three or more days, one or more weeks, one or more months, etc., taken daily, semi-daily, semi-weekly, weekly, etc.

Formulations may be optimized for retention and stabilization in the brain. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

The invention may be better understood with reference to the accompanying examples.

### EXPERIMENTAL

It was found by the inventors that short-term (4 weeks) blockade of P-selectin or the newly described P-selectin ligand TIM-1, even when given at mid to late-stage disease well after behavioral changes are observed, resulted in a profound blockade of cognitive decline. One month following treatment period, control-antibody treated AD mice showed significant cognitive impairment in two standard tests (the Y-maze spontaneous alternation task to measure special working memory and the contextual fear conditioning to measure hippocampus-dependent form of memory). In contrast, anti-P-selectin and anti-TIM-1 treated mice showed significantly reduced impairment, performing equally well in both tests compared to age-matched normal healthy mice. It is important to note that treatment was initiated at late-stage (9 months) of disease, well after behavioral changes were evident. The control-antibody-treated mice showed significant further cognitive decline at 11 months, as expected in this model, whereas the anti-P-selectin-treated and anti-TIM-1-treated mice showed cognitive impairment compared to healthy, normal age-matched mice in the fear conditioning test and Y-maze test, compared to the control-antibody-treated mice.

### Example 1

### Neutrophils Arrest and Spread on TIM-1 Glycoprotein Under Physiological Flow Conditions

We discovered that neutrophils adhere to and spread on TIM-1 under flow conditions. TIM-1 was purchased from Sino Biological Inc. Neutrophils (PMNs) were isolated from mouse bone marrow using standard methods and performed flow adhesion assays with the BioFlux microfluidic system, in which PMNs were fluxed under a physiological flow of 1 dyne/cm² in wells pre-coated with 15 μg/ml of recombinant mouse TIM-1. We found that TIM-1 was able to capture PMNs under physiological flow conditions in a divalent cation dependent manner (Table 1). Strikingly, while most of the cells undergoing immediate firm arrest, stop-and-go as well as rolling are also observed showing that TIM-1 is involved in multiple adhesion behaviors potentially involving distinct ligands and/or activation of integrins or other adhesion pathways. Strikingly, several PMNs rapidly spread immediately after arrest, showing a rapid neutrophil activation following interaction with TIM-1. Data are mean and standard deviation, P<0.05 for each behavior comparing EDTA treated vs control neutrophils.

TABLE 1
TIM-1 can arrest and spreading of neutrophils under flow conditions

| | |
|---|---|
| | EDTA |
| Control | (10 mM) |

| | Mean | SD | MEAN | SD |
|---|---|---|---|---|
| Stop and go | 23.63 | 5.37 | 1.00 | 1.00 |
| Rolling | 22.00 | 10.00 | 1.00 | 1.00 |
| Immediate Arrest | 77.25 | 12.94 | 2.00 | 2.00 |
| Spreading | 10.63 | 7.74 | 1.00 | 1.00 |

### Example 2

### Interaction of Neutrophils with TIM-1 Involves the IgV and Mucin Domain on TIM-1

In this example we show that neutrophil interaction with TIM-1 under physiological flow conditions involves the IgV and the mucin domain. Neutrophil isolation and flow assays were performed as described in Example 1. In example 1 we showed that neutrophils roll, arrest and spread on TIM-1 under physiological flow conditions. We next assessed the molecular basis of neutrophil interactions with TIM-1 under physiological flow conditions. First we compared the behavior of the cells on the IgV domain and the mucin domain compared to the full-length molecule. Flow assays were performed as described in example 1 using isolated bone marrow neutrophils on wells pre-coated with full length recombinant TIM-1, Tim-1 IgV domain, or TIM-1 mucin domain fusion proteins (all at 15 ug/ml). The data (Table 2) shows that the full length and IgV domain are capable of supporting capture, arrest and spreading under flow. Interaction with the mucin domain is reduced compared to the full-length and IgV domains; however, stop-and-go and immediate arrest on the mucin domain is observed showing that mucin domain is also capable of supporting neutrophil adhesion (but not rolling or spreading).

TABLE 2
Both full-length and the IgV domain of TIM-1 are able to support capture,
arrest and spreading of neutrophils under flow; the mucin domain supports
stop & go and immediate arrest.

| | | | TIM-1 | | | |
|---|---|---|---|---|---|---|
| | TIM-1 full | | IgV bacteria | | TIM-1 mucin | |
| | mean | SD | mean | SD | mean | SD |
| stop & go | 10.4 | 3.3 | 10.6 | 9.9 | 3.6* | 3.0 |
| rolling | 16.6 | 9.6 | 5.0 | 1.3 | 0.3* | 0.5 |
| immediate arrest | 64.0 | 22.0 | 50.0 | 16.0 | 9.5* | 3.8 |
| spreading | 18.6 | 12.5 | 10.0 | 3.2 | 0.3* | 0.5 |

*P < 0.05 compared to TIM-1 full length molecule

### Example 3

### Interactions of Neutrophils with TIM-1 Involves the Adhesion Molecules PSGL-1 and LFA-1

Example 1 showed that TIM-1 supports the adhesion of neutrophils underflow. Strikingly, TIM-1 is able to support stop- and go, modest rolling and immediate arrest of neutrophils, which generally involve multiple adhesion pathways. Further, some PMNs rapidly spread immediately after arrest, showing a rapid neutrophil activation, possibly involving integrin activation, following interaction with TIM-1. In this example we assessed the contribution of PSGL-1 and LFA-1 on binding of neutrophil adhesion behavior to full-length TIM-1. Table 3 shows that pre-incubation of the neutrophils with anti-PSGL-1 (clone 4RA10, 250 micrograms/ml) blocks stop-and-go, rolling, arrest and spreading and blockade of LFA-1 using anti-LFA-1 (TIB213 clone, 250 micrograms/ml) reduces arrest and spreading. The data show that the interaction of neutrophils with TIM-1 under physiological flow conditions involves the P-selectin ligand PSGL-1 as well as the integrin LFA-1.

TABLE 3
Neutrophil interaction with TIM-1 under flow is blocked by anti-PSGL-1
and anti-LFA-1 MAbs

| | | |
|---|---|---|
| CTRL | anti-PSGL-1 | anti-LFA-1 |

| | mean | SD | mean | SD | mean | SD |
|---|---|---|---|---|---|---|
| stop & go | 9.6 | 4.3 | 2.3* | 2.1 | 4.1 | 2.4 |
| rolling | 8.6 | 5.7 | 2.4* | 2.1 | 5.7 | 2.7 |
| immediate arrest | 44.9 | 9.1 | 18.9* | 5.0 | 15.1* | 7.7 |
| spreading | 12.1 | 9.7 | 5.6* | 4.3 | 3.7* | 2.1 |

P < 0.05 compared to control

The involvement of both PSGL-1 and LFA-1 in neutrophil interaction with TIM-1 was confirmed in flow assays using neutrophils lacking PSGL-1 (isolated form PSGL-1 knock-out mice) or LFA-1 (isolated from LFA-1 knockout mice). Neutrophils lacking PSGL-1 (Table 4, KO-PSGL-1) and neutrophils lacking LFA-1 (Table 5, KO-LFA-1) had reduced interaction, including stop and go, rolling, immediate arrest and spreading, P<0.05) with TIM-1 compared to WT neutrophils.

TABLE 4
Reduced TIM-1 interaction by PSGL-1 deficient neutrophils

| | |
|---|---|
| WT | KO - PSGL-1 |

| | mean | SD | mean | SD |
|---|---|---|---|---|
| stop & go | 23.6 | 5.4 | 7.7* | 4.4 |
| rolling | 20.0 | 10.0 | 6.3* | 6.8 |
| immediate arrest | 77.3 | 12.9 | 16.1* | 6.4 |
| spreading | 10.6 | 7.7 | 1.9* | 1.9 |

TABLE 5
Reduced TIM-1 interaction by LFA-1 deficient neutrophils

| | |
|---|---|
| WT | KO - LFA-1 |

| | mean | SD | mean | SD |
|---|---|---|---|---|
| stop & go | 31.3 | 4.5 | 14.3* | 8.6 |
| rolling | 115.4 | 35.7 | 47.7* | 28.3 |
| immediate arrest | 269.6 | 89.0 | 26.9* | 20.9 |
| spreading | 16.3 | 17.5 | 2.9* | 3.3 |

Together, the data in examples 1-3 show that TIM-1 mediates multiple steps in neutrophil adhesion, including tethering/stop- and go, rolling, immediate arrest, activation and spreading and involves both the IgV and mucin domains. Further, the data show that multiple ligands and adhesion pathways, including adhesion molecules TIM-1, PSGL-1, and LFA-1 as well as an activation step are involved in TIM-1-mediated adhesion.

### Example 4

### TIM-1 is Constitutively Expressed on a Brain Endothelial Cell Line

In this example we show that TIM-1 is constitutively expressed on brain-derived endothelial cells and that TIM-1 becomes clustered upon stimulation of endothelial cells, which can increase affinity and avidity of ligand binding and increased adhesion of circulating leukocytes. We analyzed TIM-1 expression on brain endothelial cell line bEnd.3 (Watanabe T et al., Biol. Pharm. Bull. 2013) by immunofluorescence staining. We compared TIM-1 expression to other two TIM family molecules (TIM-3 and TIM-4), to VCAM-1 and to CD31 molecule, which is constitutively expressed on endothelial cells. Briefly, bEnd.3 cells were cultured to confluence on glass slides and fixed with paraformaldehyde 4%. Cells were then labeled with antibodies: anti-mouse TIM-1 (5F12, provided by Dr. V. Kuchroo), anti-TIM-3 (polyclonal goat anti-mouse TIM-3, biotin conjugated (R&D Systems), anti-TIM-4 (polyclonal rabbit anti-TIM-4, biotin conjugated, Bioss USA), anti-VCAM-1 (clone MK2.7) or CD31 (Clone: 390; eBioscience) and isotype or species-matched control antibodies, followed by biotinylated secondary antibody and avidin Texas red for detection. Finally, DAPI staining for cell nuclei was performed.

The expression of unstimulated (basal) and stimulated (stimulated with the cells were treated for 6 hours with TNF-α) to compare basal and stimulated expression. Fluorescence intensity was scored (Table 6) as follows: no staining: (−), Positive staining (+); highly positive staining (++). Further, the distribution of the staining was assessed and scored as uniform on the cell membrane (uniform) or clustered.

TABLE 6
TIM-1 is constitutively expressed on a brain endothelial cell line

| | | TNF-stimulated (10 ng/ml |
|---|---|---|
| MAb | Basal | for 6 hours) |
| Control antibody | − | − |
| TIM-1 (5F12) | + uniform | + clustered |
| TIM-3 | − | − |
| TIM-4 | − | − |
| VCAM-1 | − | ++ uniform |
| CD31 | + uniform | + uniform |

Surprisingly, we detected constitutive TIM-1 expression on bEnd.3 cells, while the other TIM family members TIM-3 and TIM-4 protein were not present (Table 6). Stimulation bEnd.3 cells with murine TNF-α 10 ng/ml induced up-regulation of VCAM-1, whereas TIM-1 expression appeared to be reorganized on the cell surface after TNF-α treatment, clustering in specific areas on the cell membrane.

### Example 5

### Anti-TIM-1 Therapy at Mid-Late Stage Disease Reduces Cognitive Impairment in a Mouse Model of AD

In this example we show that blockade of TIM-1 has therapeutic effect in 3xTg animal model of Alzheimer's disease presenting both human amyloid and tau pathology. The role of the TIM-1 was tested using anti-TIM-1 antibody (RMT1-10, Bioxcell, CA) blockade in the 3xTG mouse model of AD. Control (anti-RAS) and anti-TIM-1 mAb therapy was initiated at 9 months, when significant cognitive impairment was evident compared to wild-type (WT) age-matched control animals using in two standard cognitive tests (the Y-maze spontaneous alternation task to measure special working memory and the contextual fear conditioning to measure hippocampus-dependent form of memory). Treatment continued for 4 weeks and then mice were allowed to recover from the repeated handling for 4 weeks and testing was performed at 11 months.

Briefly, the mAbs were diluted into sterile endotoxin-free PBS. mAbs were injected intraperitoneally at a dose of 0.5 mg per mouse for the first treatment. Then, mice were injected with 250 μg of mAbs i.p. every second day. The total volume containing the mAb was 2001□l. The treatment was continued for 4 weeks. One month following the end of antibody treatment, mice were tested in the contextual fear-conditioning test (measured as percent freezing; Table 7) and the Y-maze spontaneous alternation test (measured as % alternation; Table 7). Hind limb clasping and ledge tests were first performed to assess mice motor coordination in order to determine if alterations in vestibular function might potentially cause difficulties during behavioral assessment. Treated and not-treated 3xTg mice did not perform significantly worse compared with age-matched non-transgenic control mice showing that the differences seen in the cognitive tests are not due to differences in motor coordination or vestibular function.

The Y Maze Spontaneous Alternation is a behavioral test used to evaluate, without training, reward, or punishment, the willingness of rodents to explore new environments and to assess hippocampus-dependent spatial working memory, which is classified as short-term memory. Testing occurs in a Y-shaped maze with three gray opaque plastic arms at a 120° angle from each other, extending from a central space. Mice are introduced to a novel maze and allowed to freely explore the maze for 8 minutes. Rodents typically prefer to investigate a new arm of the maze rather than returning to one that was previously visited. The sequence and the total number of arm entries were recorded in order to calculate the percentage of alternation. Alternation was defined as successive entries into three different arms of the maze.

We also performed contextual fear conditioning test, a useful tool to study hippocampus-dependent form of memory In mice, the expression of the fear memory is illustrated by freezing behavior, a lack of movement except the one necessary for breathing. In this test paradigm the associative learning of a neutral cue (sound tone) with a brief aversive stimulus (mild electric shock) is measured by monitoring the freezing behavior in mice. The fear-conditioning test was performed by placing a mouse in a box equipped with a camera for monitoring and recording the freezing behavior of the animal. The mice were first trained to associate the sound pulse with the mild electric shock, and no significant differences in freezing behavior were obvious among mice during the training period. Then, cue-dependent freezing was tested in a novel environment (one with different lighting, olfactory and visual cues) and the freezing behavior associated with the tone was measured. Wild-type control mice exhibited a robust freezing in response to the sound tone (Table 7, percent freezing), whereas, in contrast, Ras-treated-5XFAD mice were significantly impaired (Table 7, percent freezing).

TABLE 7
Anti-TIM-1 therapy at mid-late stage disease reduces loss of cognitive
impairment in a model of Alzheimer's Disease

| | | | P value* | | | P value* |
|---|---|---|---|---|---|---|
| | % Freezing | | compared to | % Alternation | | compared |
| | Mean | SEM | WT | Mean | SEM | to WT |
| WT | 45.1 | 2.6 | | 68 | 2 | |
| Control Mab | 30.1 | 2.9 | P < 0.005 | 58 | 3 | P < 0.005 |
| Anti-TIM-1 | 40.0 | 3.2 | NS | 67 | 5 | NS |

*P values calculated using a one-tailed unpaired t test
**P < 0.05 for control Mab vs anti-TIM-1 treated mice

The contextual fear-conditioning test is a useful tool to study hippocampus-dependent form of memory. Wild-type control mice exhibited a robust freezing in response to the sound tone (Table 7, percent freezing), whereas, in contrast, Control Mab (anti-Ras)-treated mice were significantly impaired (P<0.001 compared to WT age-matched controls, Table 7, percent freezing). Treatment with anti-TIM-1 antibody resulted in a significant reduction of the memory impairment (no significant difference compared to wild-type age-matched control mice). The Y-maze test is a useful tool to study hippocampus-dependent spatial working memory, Control mab-treated animals displayed significantly reduced hippocampus-dependent spatial working memory (percent alternation; Table 7) which is classified as short-term memory. Compared to wild-type control animals; whereas Anti-TIM-1-treated animals performed as well as the wild-type animals, displaying no impairment in short-term memory.

### Example 6

### Anti-TIM-1 Therapy Reduces Amyloid Deposition, Phosphorylated Tau and Microglia Activation in a Model of AD

In this example we show that treatment of AD mice with anti-TIM-1 Mab for 4 weeks starting at 9 months of age, when both proteins are increased above normal in this model, resulted in a significant reduction of both beta amyloid deposition and phosphorylated tau. 3x-TG-AD mice were treated with anti-TIM-1 antibody RMT1-10 or an isotype-matched control antibody for 4 weeks starting at 9 months of age. After a one month washout period mice were tested in cognitive testing assays (see example 5) and sacrificed. Sections were obtained from the anterior hippocampus through the bregma −2.9 mm at intervals of 500 micrometers in order to analyze the entire hippocampus. The amount of beta-amyloid and phosphorylated tau in the CA1 region of hippocampus was assessed using blinded quantitative stereological analysis of sections stained with an antibody specific for beta amyloid (6E10 antibody; Covance), phosphorylated tau Thr231 (At180 Mab, Thermo Scientific) or isotype matched control MAbs. Secondary antibody was a biotinylated goat anti-mouse Mab (Sigma) and immunoreactivity was visualized using the VECTASTAIN ABC kit and Vector NovaRED (Vector) reagents. Images were acquired using fluorescence microscopy and counted blindly with ImageJ v1.32j software. Both beta-amyloid and phosphorylated tau were significantly decreased after 4 weeks of anti-TIM-1 therapy and a one-month washout period compared to control-treated AD mice. Total tau was unchanged.

Microglia are the resident macrophages of the brain and spinal cord and can be identified by various markers including Iba-1. Histological evaluation of the microglial cells in brain, using Iba-1+ as a marker of microglial, cells, revealed a highly activated phenotype of microglial cells, defined as cells with an enlarged cell body and thick, retracted processes, in the control-antibody treated AD mice as compared to unactivated microglial cells, defined as cells with small, round soma and long processes, in neutrophil/myeloid depleted mice.

The numerical density of Iba-1⁺ immunoreactive microglia was determined in 4 non-consecutive coronal sections throughout the cortex and the dorsal hippocampus of both 3xTg-AD and WT control mice. The specific analyzed areas were the parietal cortex, the dentate gyrus and the CA1 hippocampus. Iba-1⁺ microglia cells were visualized using a LEICA fluorescence microscope (DM6000B, Leica) and counted blindly with ImageJ 1.32j software. Unbiased quantitative stereologic analysis was performed on cortex slices to determine the total number and area of Iba-1+ cells (Table 9). Microglia cell density and area was lower in TIM-1 Mab-treated animals (Table 9). In addition, the microglial cells displayed a non-activated phenotype in TIM-1-Mab treated AD mice compared to isotype-control treated AD mice.

TABLE 8
Anti-TIM-1 therapy reduces beta-amyloid deposition and
phosphorylated tau in the hippocampus in a mouse model of AD

| | Isotype mAb | Anti-TIM-1 mAb |
|---|---|---|
| Beta-amyloid | 248,827 ± 28,797* | 139,718 ± 22,880* |
| Phosphorylated Tau | 148,200 ± 27,390** | 57,220 ± 9519** |

Data are the number of pixels/0.4 × 0.3 mm area; mean and SEM
*and **P < 0.005; Statistical analysis was the Mann-Whitney test.

TABLE 9
Anti-TIM-1 therapy reduces the number and area
of microglia in the brain in a mouse model of AD

| | Isotype Control | Anti-TIM-1 | P value |
|---|---|---|---|
| Area | 9689 | 7111 | 0.0022 |
| Density | 38 | 28 | 0.0016 |

### Example 7

### Anti-P-Selectin Therapy Reduces Cognitive Impairment in a Mouse Model of AD

In this example we show that blockade of P-selectin has therapeutic effect in 3xTg animal model of Alzheimer's disease presenting both human amyloid and tau pathology and that therapeutic targeting of P-selectin represents a new therapeutic approach for Alzheimer's disease.

The role of the P-selectin adhesion pathway was tested using anti-P-selectin antibody (clone RB40 from Bioxcell, CA) blockade in the 3xTG mouse model of AD. Anti-P-selectin treatment was initiated at 9 months, when significant cognitive impairment was evident ng both the fear conditioning and Y maze testing protocols. Treatment continued for 4 weeks and then mice were allowed to recover from the repeated handling for 4 weeks and testing was performed at 11 months.

Briefly, the mAbs were diluted into sterile endotoxin-free PBS. mAbs were injected intraperitoneally at a dose of 0.5 mg per mouse for the first treatment. Then, mice were injected with 250 μg of mAbs i.p. every other day for 4 weeks followed by a one month washout period.

One month following the end of antibody treatment, mice were tested in the contextual fear-conditioning test (measured as percent freezing; Table 10) and in the Y-maze spontaneous alternation task (percent alternation; Table 11). To rule out any issues with vestibular function that might cause difficulties during behavioral assessment, hind limb clasping and ledge tests were first performed to assess mice motor coordination. No differences in motor coordination were detected comparing treated and not-treated 3xTg mice.

The contextual fear-conditioning test is a useful tool to study hippocampus-dependent form of memory. The expression of the fear memory is illustrated by freezing behavior, a lack of movement except the one necessary for breathing. In this test paradigm the associative learning of a neutral cue (sound tone) with a brief aversive stimulus (mild electric shock) is measured by monitoring the freezing behavior in mice. The fear-conditioning test was performed by placing a mouse in a box equipped with a camera for monitoring and recording the freezing behavior of the animal. The mice were first trained to associate the sound pulse with the mild electric shock, and no significant differences in freezing behavior were obvious among mice during the training period. Then, cue-dependent freezing was tested in a novel environment (one with different lighting, olfactory and visual cues) and the freezing behavior associated with the tone was measured. Wild-type control mice exhibited a robust freezing in response to the sound tone (Table 10, percent freezing), whereas, in contrast, Control Mab (anti-human Ras, Y13259 clone)-treated mice were significantly impaired (P<0.005, Table 10, percent freezing). Treatment with anti-P-selectin antibody resulted in a significant reduction of the memory impairment whereas no significant difference compared to wild-type age-matched control mice.

TABLE 10
Anti-P-selectin therapy reduces hippocampus-dependent
memory loss in a model of Alzheimer's disease

| | % Freezing | | |
|---|---|---|---|
| Fear Conditioning | Mean | SEM | P value* compared to WT |
| WT | 46.8 | 4.2 | |
| Control Mab | 29.4** | 4.1 | P < 0.005 |
| Anti-P-selectin | 38.2** | 2.9 | NS |

*P values calculated using a one-tailed unpaired t test
**P < 0.05 for control Mab vs anti-P-selected treated mice

TABLE 11
Anti-P-selectin therapy reduces loss of spatial working
memory in a mouse model of Alzheimer's disease

| | |
|---|---|
| | P value* |
| % Alteration | compared |

| Y-Maze | Mean | SEM | to WT |
|---|---|---|---|
| WT | 64.6 | 2.1 | |
| Control Mab | 52.5* | 1.6 | P < 0.0005 |
| Anti-P-selectin | 59.3* | 2.1 | NS |

*unpaired one-tailed t test
**Anti-P-selectin vs control Mab P < 0.01

The Y-maze spontaneous alternation task measures spatial working memory, and exploits the natural tendency of rodents to explore novel locations. Mice with intact spatial working memory are more likely to enter and explore an arm that have not recently visited rather than one that is familiar, and therefore have higher levels of spontaneous alternation. Mice making fewer than 15 total arm entries during the 8 min test were excluded from groups, in order to avoid that low numbers of entries may affect the spontaneous alternation score. The number of arms entered during the test was comparable among AD mice treated with anti-neutrophil or control antibodies and control wild-type age-matched mice, indicating that AD mice had normal motor function and exploratory activity. As expected, a significantly decreased alternation in Y-maze task was detectable in anti-Ras-treated (control antibody) mice compared to control wild-type age-matched mice (Table 10). Surprisingly, treatment of 3xT mice with anti-P-selectin starting at 9 months of age, after significant cognitive impairment has already occurred, significantly reduced the cognitive deficit and mice performed at comparable levels of control wild-type healthy age-matched littermates.

### REFERENCES

Angiari S et al., 2014, TIM-1 glycoprotein binds the adhesion receptor P-selectin and mediates T cell trafficking during inflammation and autoimmunity., Immunity 40(4):542-553
Angiari and Constantin (2014) Regulation of T cell trafficking by the T cell immunoglobulin and mucin domain 1 glycoprotein. Trends Mol Med. 20(12):675-84
Binné, L. L. et al. (2007) Human TIM-1 associates with the TCR complex and up-regulates T cell activation signals. J. Immunol. 178, 4342-4350.
Bonventre, J. V. 2014, Kidney injury molecule-1: a translational journey Trans Am Clin Climatol Assoc 125; 293-9
Curtiss, M. L. et al. (2011) Fyn binds to and phosphorylates T cell immunoglobulin and mucin domain-1 (Tim-1). Mol. Immunol. 48, 1424-1431.
de Souza, A. J. et al. (2008) T cell Ig and mucin domain-1-mediated T cell activation requires recruitment and activation of phosphoinositide 3-kinase. J. Immunol. 180, 6518-6526.
Ernst B1, Magnani J L. From carbohydrate leads to glycomimetic drugs. Nat Rev Drug Discov. 2009 August; 8(8):661-77.
Frenette P S et al., 2000 P-Selectin glycoprotein ligand 1 (PSGL-1) is expressed on platelets and can mediate platelet-endothelial interactions in vivo. J. Exp Med. 2000, 191(8):1413-22
Geraldo R B et al (2014) Platelets: still a therapeutical target for haemostatic disorders. Int J Mol Sci. 15(10):17901-19
Kuchroo V K, Dardalhon V, Xiao S, Anderson A C. New roles for TIM family members in immune regulation. Nat Rev Immunol. 2008 August; 8(8):577-80.
Ley and Kansas. (2004). Selectins in T-cell recruitment to nonlymphoid tissues and sites of inflammation. Nat. Rev. Immunol. 4, 325-335.
Ley, K., Laudanna, C., Cybulsky, M. I., and Nourshargh, S. (2007). Getting to the site of inflammation: the leukocyte adhesion cascade updated. Nat. Rev. Immunol. 7, 678-689.
Lleo et al, 2015. Cerebrospinal fluid biomarkers in trials for Alzheimer and Parkinson diseases. Nat Rev Neurol. January; 11(1):41-55
Moller-Tank and Maury, 2014 Phosphatidylserine receptors: enhancers of enveloped virus entry and infection. Virology 468-70, 565-580, 2014
Oddo et al., 2003 Triple-transgenic model of Alzheimer's disease with plaques and tangles: intracellular Abeta and synaptic dysfunction. Neuron: 39(3):409-21
Rodriguex-Manzanet R., et al., 2009, The costimulatory role of TIM molecules. Immunol. Rev., 229: 259-270
Wang, H. B. et al. (2007) P-selectin primes leukocyte integrin activation during inflammation. Nat. Immunol. 8, 882-892.
Watanabe T (2013) Paracellular barrier and tight junction protein expression in the immortalized brain endothelial cell lines bEND.3, bEND.5 and mouse brain endothelial cell 4. Biol Pharm Bull. 36(3):492-5.
Wilker P R et al (2007) Evidence for carbohydrate recognition and homotypic and heterotypic binding by the TIM family. Int Immunol. 19(6):763-73.
Evidence for carbohydrate recognition and homotypic and heterotypic binding by the TIM family.
Zenaro E, et al (2015) Neutrophils promote Alzheimer's disease-like pathology and cognitive decline via LFA-1 integrin. Nat Med. 2015 21(8):880-6.
Freeman G J, et al., 2010, TIM genes: a family of cell surface phosphatidylserine receptors that regulate innate and adaptive immunity. Immunol. Rev, 235: 172-189
Wilker P R, 2007, Evidence for carbohydrate recognition and homotypic and heterotypic binding by the TIM family. Int. Immunol., 19: 762-773
Zarbock, A., Ley, K., McEver, R. P., and Hidalgo, A. (2011). Leukocyte ligands for endothelial selectins: specialized glycoconjugates that mediate rolling and signaling under flow. Blood 118, 6743-6751

What is claimed is:

1. A method of prevention and treatment of neurodegenerative disease in an individual mammal, said method comprising:
administering to said individual mammal an effective amount of an agent that inhibits TIM-1 activity in the region of the brain.

2. The method of claim 1, wherein the agent inhibits a P-selectin pathway.

3. The method of claim 1, wherein the agent that inhibits TIM-1 is administered in combination with an agent that inhibits a P-selectin pathway.

4. The method of claim 1, wherein the agent that inhibits TIM-1 binds to one or more of the TIM-1 IgV domain, mucin domain, sialic acid binding site, PSGL-1 binding site, P-selectin binding site, LFA-1 binding site.

5. The method of claim 1, wherein the agent that inhibits TIM-1 or the agent that inhibits P-selectin pathway is an antibody or fragment thereof.

6. The method of claim 1, wherein the agent that inhibits TIM-1 or the agent that inhibits P-selectin pathway is a glycan or glycomimetic.

7. The method of claim 1, wherein the agent that inhibits TIM-1 or the agent that inhibits P-selectin pathway is a peptide or peptidomimetic.

8. The method of claim 1, wherein the agent that inhibits TIM-1 or the agent that inhibits P-selectin pathway is small molecule.

9. The method of claim 1, wherein the neurodegenerative disease is selected from Alzheimer's disease, Parkinson's disease, Huntington's disease, amyotrophic lateral sclerosis, vascular dementia, dementia or mild cognitive impairment.

10. The method of claim 1, wherein the treatment reduces development of cognitive deficits in the mammal.

11. The method of claim 1, wherein the individual is diagnosed prior to treatment.

12. The method of claim 1, wherein the individual is differentially diagnosed with AD.

13. The method of claim 1, wherein the agent is administered systemically.

14. The method of claim 13, wherein the agent does not cross the blood brain barrier after administration.

15. The method of claim 1, wherein the agent is administered intrathecally or locally to a region of the brain.

16. The method of claim 1, wherein monitoring of disease progression is performed at multiple time points by analysis of one or more of the presence of biomarkers, cognitive testing, imaging, amyloid deposits, and activation of microglia.

17. The method of claim 1 wherein the agent that inhibits TIM-1 blocks one or more stages of leukocyte adhesion and migration.

18. A kit for use in the methods of claim 1, comprising an agent and instructions for use.

19. A unit dose of a medicament for us in the methods of claim 1.
